(19) United States
(12) Patent Application Publication
(10) Pub. No.: US 2026/0294450 A1
(43) Pub. Date: Oct. 1, 2026

(54) BARBED GUIDE WIRE FOR TREATMENT OF PLASTIC BRONCHITIS

(71) Applicant: Shengjing Hospital of China Medical University, Shenyang (CN)

(72) Inventors: Wei Xu, Shenyang (CN); Hongzan Sun, Shenyang (CN); Jiajun Wang, Shenyang (CN)

(21) Appl. No.: 19/205,968

(22) Filed: May 12, 2025

(30) Foreign Application Priority Data
Mar. 31, 2025 (CN) 202510399097.6

(51) Int. Cl. A61B 17/22; A61B 17/00

(52) U.S. Cl. CPC A61B 17/22; A61B 2017/00367; A61B 2017/00477; A61B 2017/00809; A61B 2017/22042

(57) ABSTRACT

Disclosed is a barbed guide wire for treatment of plastic bronchitis, which relates to the technical field of medical devices. The barbed guide wire for treatment of plastic bronchitis provided by the present disclosure can be directly inserted into a bronchial cast through a sleeve, then barbs expand to hook the bronchial cast and pull back by pushing the guide wire, such that the bronchial cast can be taken out completely, to prevent the bronchial cast breaking off and remaining in a distal bronchus, prevent from affecting ventilation, and reduce a risk of bronchoalveolar lavage due to the breakage of the bronchial cast.

### CROSS-REFERENCE TO RELATED APPLICATION

This application claims priority of Chinese Patent Application No. 202510399097.6, filed on Mar. 31, 2025, the entire contents of which are incorporated herein by reference.

### TECHNICAL FIELD

The present disclosure relates to the technical field of medical devices, and in particular, to a barbed guide wire for treatment of plastic bronchitis.

### BACKGROUND

Pediatric plastic bronchitis (PB) is a rare but life-threatening disease characterized by the formation of casts within a tracheobronchial tree, leading to partial or complete obstruction of the bronchi, causing partial or total ventilation dysfunction, and seriously causing respiratory insufficiency or even suffocation. The treatment of pediatric plastic bronchitis requires the identification of the lesion site under pulmonary imaging guidance, followed by bronchoscopic removal of the casts.

Currently, the most effective and direct treatment for pediatric plastic bronchitis is the removal of obstructive casts via bronchoscopy. However, during the process of grasping and removing the casts, the thinner distal part of the cast is prone to breaking off and remaining in the distal bronchi, which can affect pulmonary ventilation. Consequently, bronchoalveolar lavage is often required to expel the broken and residual casts from the bronchi. Nevertheless, bronchoalveolar lavage may also introduce new complications and risks, and the extent of lung injury it may cause remains unclear.

### SUMMARY

An objective of the present disclosure is to provide a barbed guide wire for treatment of plastic bronchitis, to solve the above-mentioned problems.

To solve the above-mentioned technical problems, the present disclosure adopts the following technical solutions:

The present disclosure provides a barbed guide wire for treatment of plastic bronchitis, including a sleeve with one end sealed and a guide wire placed inside the sleeve. An opened end of the sleeve is connected to a first gripping portion, a second gripping portion is connected to one end, placed outside the sleeve, of the guide wire, and the other end of the guide wire is connected to an inner wall of the sealed end of the sleeve through a miniature pressing self-locking buckle. The guide wire is provided with multiple groups of barb assemblies on an outer wall of one end inside the sleeve. Each of the barbed assemblies includes two symmetrically arranged barbs, which are placed in a placement hole in a sleeve body. One end of each of the barbs is hinged on an inner wall of the sleeve, and the barbs are controlled to expand by a push assembly connected to the guide wire.

Further, the second gripping portion is in a circular ring shape, and the second gripping portion is hinged to an end part of the guide wire.

Further, seven groups of barb assemblies are connected to an outer wall of the end part of the guide wire, with equal spacing between adjacent groups of barb assemblies.

Further, a plurality of spikes are disposed on a sealed end face, deviating from the sleeve, of the barb.

Further, the push assembly includes a push plate connected to the guide wire; two connecting blocks that correspond to positions of two barbs in respective are connected to the push plate, a first push rod is connected to the connecting blocks, and the other end of the first push rod is connected with a first baffle after being inserted into a J-shaped special-shaped tube; the special-shaped tube includes a long straight section, an arc-shaped section, and a short straight section; the first baffle is slidingly connected inside the long straight section. A first spring is connected to one end face, away from each of the connecting blocks, of the first baffle, the first spring is placed inside the arc-shaped section, and the other end of the first spring is connected to a second baffle slidingly connected inside the short straight section; a second push rod is connected to one end face, away from the first spring, of the second baffle, the other end of the second push rod is placed outside the special-shaped tube and connected to a push block, and the push block is pressed against end parts of the barbs.

Further, the first baffle and the second baffle are both slidingly connected in the special-shaped tube through a sliding rail mounted on an inner wall of the special-shaped tube.

Further, a jack is formed in an inner wall of the placement hole, the jack communicates with a cavity disposed in a tube wall of the sleeve, a third baffle is inserted in the jack, the third baffle is placed inside the barbs at one end placed outside, the other end of the third baffle is placed in the cavity and connected to a pressing plate, and a second spring is disposed between the pressing plate and an inner wall of the cavity.

Further, the sleeve is a flexible tube.

Further, a mounting slot is formed in an outer wall of the sealed end of the sleeve, a sensor is mounted in the mounting slot, and an outer wall of the sensor matches with an arc surface on the sealed end of the sleeve.

Further, the sensor is connected to an external control end through a lead embedded inside the sleeve.

Compared with the existing technology, the present disclosure has following beneficial technical effects.

The barbed guide wire for treatment of plastic bronchitis provided by the present disclosure can be directly inserted into the bronchial cast through the sleeve, then the plurality of barbs expand to hook the bronchial cast and pull back by pushing the guide wire, such that the bronchial cast can be taken out quickly and completely, to prevent the bronchial cast breaking off and remaining in the distal bronchus, prevent from affecting ventilation, and reduce a risk of bronchoalveolar lavage due to the breakage of the bronchial cast.

### BRIEF DESCRIPTION OF THE DRAWINGS

The following is a further explanation of the present disclosure with reference to the accompanying drawings.

FIG. 1 is a schematic structural diagram of a barbed guide wire for treatment of plastic bronchitis according to the present disclosure;

FIG. 2 is a cross-sectional view of a barbed guide wire for treatment of plastic bronchitis according to the present disclosure;

FIG. 3 is an enlarged view of point A in FIG. 2;

FIG. 4 is an enlarged view of point B in FIG. 2;

FIG. 5 is an enlarged view of point C in FIG. 2; and

FIG. 6 is a main view for mounting a spike.

Explanation of reference numerals: 1. Sleeve; 2. Guide wire; 3. First gripping portion; 4. Second gripping portion; 5. Miniature pressing self-locking buckle; 6. Barb; 7. Placement hole; 8. Spike; 9. Push plate; 10. Connecting block; 11. First push rod; 12. Special-shaped tube; 13. First baffle; 14. First spring; 15. Second baffle; 16. Second push rod; 17. Push block; 18. Sliding rail; 19. Jack; 20. Cavity; 21. Third baffle; 22. Pressing plate; 23. Second spring; 24. Mounting slot; 25. Sensor.

### DETAILED DESCRIPTION

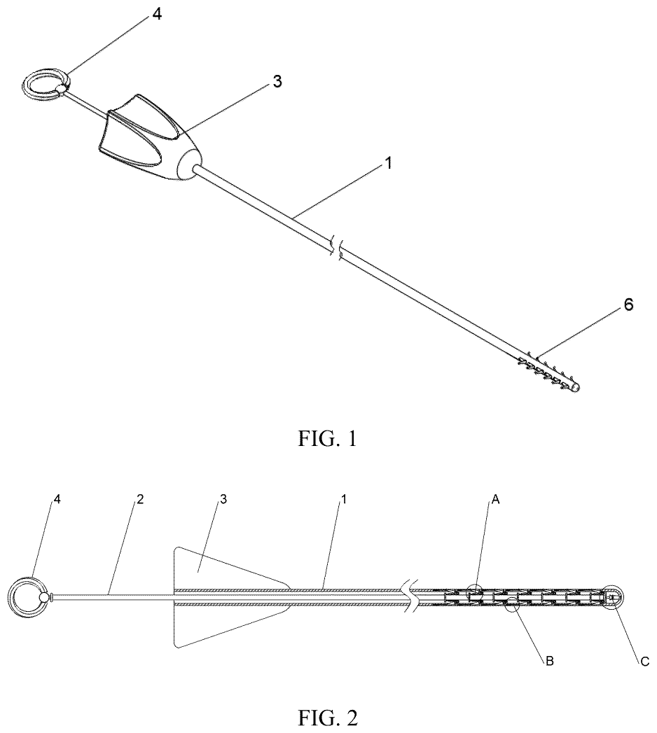

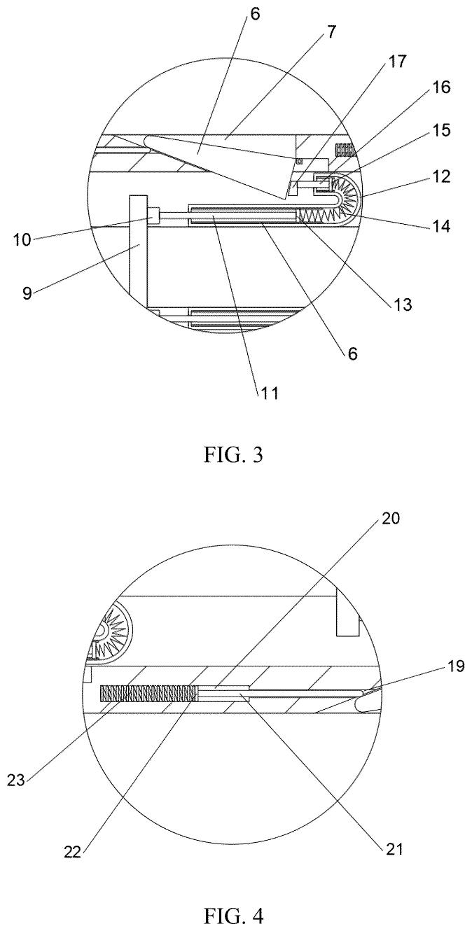

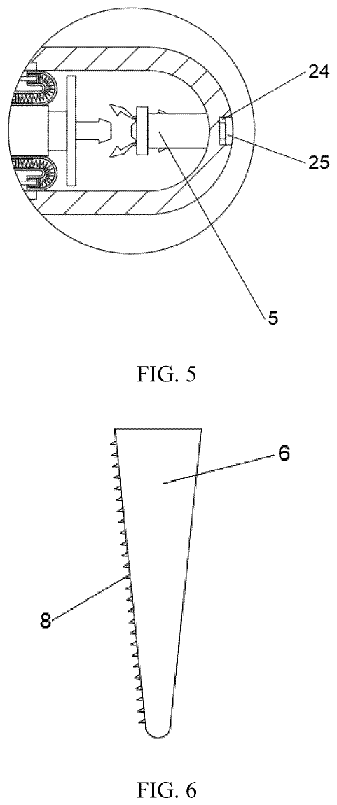

As shown in FIGS. 1-6, a barbed guide wire for treatment of plastic bronchitis includes a sleeve 1 with one end sealed and a guide wire 2 placed inside the sleeve 1. The sleeve 1 is a flexible tube.

An opened end of the sleeve 1 is connected to a first gripping portion 3, a second gripping portion 4 is connected to one end, placed outside the sleeve 1, of the guide wire 2. The second gripping portion 4 is in a circular ring shape, and the second gripping portion 4 is hinged to an end part of the guide wire 2.

The other end of the guide wire 2 is connected to an inner wall of the sealed end of the sleeve 1 through a miniature pressing self-locking buckle 5.

The guide wire 2 is provided with seven groups of barb assemblies on an outer wall of one end inside the sleeve 1, with equal spacing between adjacent groups of barb assemblies. Each of the barbed assemblies includes two symmetrically arranged barbs 6, which are placed in a placement hole 7 in a sleeve 1 body; one end of each of the barbs 6 is hinged on an inner wall of the sleeve 1, and a plurality of spikes 8 are disposed on a sealed end face, deviating from the sleeve 1, of the barbs 6.

The barbs 6 are controlled to expand by a push assembly connected to the guide wire 2.

The push assembly includes a push plate 9 connected to the guide wire 2. Two connecting blocks 10 that correspond to positions of two barbs 6 in respective are connected to the push plate 9. A first push rod 11 is connected to the connecting blocks 10, and the other end of the first push rod 11 is connected with a first baffle 13 after being inserted into a J-shaped special-shaped tube 12. The special-shaped tube 12 includes a long straight section, an arc-shaped section, and a short straight section. The first baffle 13 is slidingly connected inside the long straight section; a first spring 14 is connected to one end face, away from each of the connecting blocks 10, of the first baffle 13, the first spring 14 is placed inside the arc-shaped section, and the other end of the first spring 14 is connected to a second baffle 15 slidingly connected inside the short straight section. A second push rod 16 is connected to one end face, away from the first spring 14, of the second baffle 15, the other end of the second push rod 16 is placed outside the special-shaped tube 12 and connected to a push block 17, and the push block 17 is pressed against end parts of the barbs 6.

The first baffle 13 and the second baffle 15 are both slidingly connected in the special-shaped tube 12 through a sliding rail 18 mounted on an inner wall of the special-shaped tube 12.

A jack 19 is formed in an inner wall of the placement hole 7, the jack 19 communicates with a cavity 20 disposed in a tube wall of the sleeve 1, a third baffle 21 is inserted in the jack 19, the third baffle 20 is placed inside the barbs 6 at one end placed outside, the other end of the third baffle 21 is placed in the cavity 20 and connected to a pressing plate 22, and a second spring 23 is disposed between the pressing plate 22 and an inner wall of the cavity 20.

A mounting slot 24 is formed in an outer wall of the sealed end of the sleeve 1, a sensor 25 is mounted in the mounting slot 24, and an outer wall of the sensor 25 matches with an arc surface on the sealed end of the sleeve 1. The sensor 25 is connected to an external control end through a lead embedded inside the sleeve 1. The sensor 25 can sense a gas flowing direction, and a position where an end part is located can also be judged through the sensor 25.

The action process of the present disclosure is as follows.

When in use, the sleeve 1 is inserted into the bronchial cast, then the first gripping portion 3 is held with one hand, to push the second gripping portion 4 such that the entire guide wire 2 moves forward until a space between the end part of the guide wire 2 and the sealed end of the sleeve 1 is locked by the miniature pressing self-locking buckle 5. When the guide wire 2 is moving, the push plate 9 is allowed to push the first push rod 11, and the first spring 14 pushes the second push rod 16 to move towards one end of each of the barbs 6, such that the push block 17 pushes the barbs 6 to rotate. At this time, the third baffle 21 will come out of the jack 19 due to being not pressed by the barbs 6, to block the placement hole 7, thus preventing the bronchial cast from entering the sleeve 1. The barbs 6 are inserted into the bronchial cast after expanding, and the sleeve 1 is dragged outwards, to bring out the bronchial cast through the barbs 6.

The embodiments described above are only descriptions of the preferred modes of the present disclosure, instead of limiting the scope of the present disclosure. Without departing from the design spirit of the present disclosure, various modifications and improvements made to the technical solutions of the present disclosure by those of ordinary skill in the art should all fall within the protection scope determined by the claims of the present disclosure.

1. A barbed guide wire for treatment of plastic bronchitis, comprising a sleeve (1) with one end sealed and a guide wire (2) placed inside the sleeve (1), wherein an opened end of the sleeve (1) is connected to a first gripping portion (3), a second gripping portion (4) is connected to one end, placed outside the sleeve (1), of the guide wire (2), and the other end of the guide wire (2) is connected to an inner wall of the sealed end of the sleeve (1) through a miniature pressing self-locking buckle (5); the guide wire (2) is provided with multiple groups of barb assemblies on an outer wall of one end inside the sleeve (1), each of the barbed assemblies comprises two symmetrically arranged barbs (6), which are placed in a placement hole (7) in a sleeve (1) body; one end of each of the barbs (6) is hinged on an inner wall of the sleeve (1), and the barbs (6) are controlled to expand by a push assembly connected to the guide wire (2).

2. The barbed guide wire for treatment of plastic bronchitis according to claim 1, wherein the second gripping portion (4) is in a circular ring shape, and the second gripping portion (4) is hinged to an end part of the guide wire (2).

3. The barbed guide wire for treatment of plastic bronchitis according to claim 1, wherein seven groups of barb assemblies are connected to an outer wall of the end part of the guide wire (2), with equal spacing between adjacent groups of barb assemblies.

4. The barbed guide wire for treatment of plastic bronchitis according to claim 1, wherein a plurality of spikes (8) are disposed on a sealed end face, deviating from the sleeve (1), of the barbs (6).

5. The barbed guide wire for treatment of plastic bronchitis according to claim 1, wherein the push assembly comprises a push plate (9) connected to the guide wire (2); two connecting blocks (10) that correspond to positions of two barbs (6) in respective are connected to the push plate (9), a first push rod (11) is connected to the connecting blocks, and the other end of the first push rod (11) is connected with a first baffle (13) after being inserted into a J-shaped special-shaped tube (12); the special-shaped tube (12) comprises a long straight section, an arc-shaped section, and a short straight section; the first baffle (13) is slidingly connected inside the long straight section; a first spring (14) is connected to one end face, away from each of the connecting blocks (10), of the first baffle (13), the first spring (14) is placed inside the arc-shaped section, and the other end of the first spring (14) is connected to a second baffle (15) slidingly connected inside the short straight section; a second push rod (16) is connected to one end face, away from the first spring (14), of the second baffle (15), the other end of the second push rod (16) is placed outside the special-shaped tube (12) and connected to a push block (17), and the push block (17) is pressed against end parts of the barbs (6).

6. The barbed guide wire for treatment of plastic bronchitis according to claim 5, wherein the first baffle (13) and the second baffle (15) are both slidingly connected in the special-shaped tube (12) through a sliding rail (18) mounted on an inner wall of the special-shaped tube (12).

7. The barbed guide wire for treatment of plastic bronchitis according to claim 1, wherein a jack (19) is formed in an inner wall of the placement hole (7), the jack (19) communicates with a cavity (20) disposed in a tube wall of the sleeve (1), a third baffle (21) is inserted in the jack (19), the third baffle (20) is placed inside the barbs (6) at one end placed outside, the other end of the third baffle (21) is placed in the cavity (20) and connected to a pressing plate (22), and a second spring (23) is disposed between the pressing plate (22) and an inner wall of the cavity (20).

8. The barbed guide wire for treatment of plastic bronchitis according to claim 1, wherein the sleeve (1) is a flexible tube.

9. The barbed guide wire for treatment of plastic bronchitis according to claim 1, wherein a mounting slot (24) is formed in an outer wall of the sealed end of the sleeve (1), a sensor (25) is mounted in the mounting slot (24), and an outer wall of the sensor (25) matches with an arc surface on the sealed end of the sleeve (1).

10. The barbed guide wire for treatment of plastic bronchitis according to claim 9, wherein the sensor (25) is connected to an external control end through a lead embedded inside the sleeve (1).
